(19) United States
(12) Patent Application Publication
(10) Pub. No.: US 2018/0369022 A1
(43) Pub. Date: Dec. 27, 2018

(54) EAR-CLEANING IMPLEMENT AND METHOD

(71) Applicant: Ali Adnan KHAZAAL, Dearborn Heights, MI (US)

(72) Inventor: Ali Adnan KHAZAAL, Dearborn Heights, MI (US)

(21) Appl. No.: 15/633,965

(22) Filed: Jun. 27, 2017

(51) Int. Cl. A61F 11/00; A61F 13/38

(52) U.S. Cl. CPC A61F 11/006; A61F 13/38

(57) ABSTRACT

An ear-cleaning implement with an applicator for inserting into an ear canal. The distal end region of the applicator is provided with a rounded leading surface. At the proximal end region there is an aperture that receives a post that can be grasped between the fingers. There is a compliant middle region which has an un-deflected configuration before insertion into the ear canal. Before the implement is inserted, it is preferably squeezed between the fingers. During passage into the ear canal, after positioning therein, and upon at least partial withdrawal therefrom, the compliant middle region has at least a partially compressed configuration from which it expands to conform with the ear canal. Upon withdrawal, the implement effectively and safely removes wax from the ear canal.

### BACKGROUND OF THE INVENTION

### (1) Field of the Invention

This disclosure relates to an ear-cleaning implement and method for its use.

### BRIEF SUMMARY OF THE INVENTION

If wax accumulates in an ear canal, effective examination of the eardrum may be impeded and hearing may be adversely affected. Sometimes, the layman may try to remove the wax, for example with a cotton swab. But the cotton swab tends to act as a plunger and may cause impaction or injury to the ear drum. A cotton swab may push the wax deeper into the ear canal, may mix it with cotton fibers and dead skin cells plus environmental debris, and thus create “cerumen impaction”. Such a condition may result in hard plugs that may be difficult and painful for the layman to remove.

Earwax or cerumen is a substance produced by the skin of the outer portion of the ear. This substance protects the wall of an otherwise delicate ear canal from unwanted moisture, dehydration and bacterial infection. Sometimes, excessive ear wax may reduce the effectiveness of hearing aids.

The most commonly used ear canal hygiene method presently in use today is a cotton swab which in general may be effective to remove water and some particulates from the ear. But it is suboptimal in removing ear wax and excess ear debris. Ear wax and ear debris may be smeared in situ instead of being removed. If compacted, later removal may be difficult. Thus, one problem with cotton swabs in efforts to remove ear wax is that they may cause the wax to penetrate more deeply into the ear without actually removing the wax.

Among the art considered before filing this patent application are the following references: U.S. Pat. Nos. 4,820,259; 5,715,850; 5,888,199; 8,099,295; 2005/0096678; 2007/0299457; 2008/0142385; 2011/0066172; 2015/0128532; and 2017/0024541.

### SUMMARY

An object of the present disclosure is to provide an optionally disposable, improved ear-cleaning implement and method for its use in a human or other animal anatomy that avoids some of the drawbacks of prior approaches. An important goal of an ear cleaner is to prevent ear drum injury while providing ease of use for laypersons and professionals. What is needed and is offered by the implement described herein is a way to safely and effectively clean the ear canal while increasing the layman's ability to remove unwanted ear wax without perforating an ear drum.

In one embodiment, there is disclosed an ear-cleaning implement preferably with a shape-memory applicator that is generally sphero-cylindrical and is progressively inserted into an ear canal. As used herein, the term “sphero-cylindrical” means generally cylindrical with a hemispherical leading portion. Optionally the applicator is frusto-conical, preferably with a leading portion that is hemispherical. The applicator has a distal end region, a proximal end region and a compliant middle region therebetween.

The distal end region is provided with a rounded leading surface. At the proximal end region there is an aperture.

Between the end regions there is a compliant middle region preferably made of a shape memory material which has an un-deflected configuration before insertion into the ear canal. Before the implement is inserted, it is optionally squeezed between the fingers. During passage into the ear canal, after positioning therein, and upon at least partial withdrawal therefrom, the compliant middle region has a compressed configuration. One characteristic is that at least a portion of the compliant middle region reverts to or towards the un-deflected configuration in the ear canal after digitally applied compressive forces are removed and the applicator is at least partially withdrawn from the ear canal. During reversion to the un-deflected configuration, some accumulated debris may be shed from the implement, but most adheres to the applicator and is conveniently removed from the ear.

Preferably, the applicator has a gentle, non-aggressive adhesive surface that does not impede insertion and cause the implement to bind against the ear wall during insertion. Instead the adhesive surface guides the implement during passage into the ear canal and withdrawal therefrom.

Over these positional states, ear wax is gathered on the adhesive surface from the ear canal, the wax adhering to at least a portion of the compliant middle region upon withdrawal.

An axial bore extends from the aperture in the distal end region into the compliant middle region. Disposed within the bore is a post that is engaged by the bore so that control can be exercised by a user's fingers in positioning the shape memory applicator by fore-aft or twisting movement or both.

### BRIEF DESCRIPTION OF THE DRAWINGS

The invention and its advantages appear in greater detail in the context of the following description of embodiments given by way of illustration and with reference to the accompanying figures, in which:

FIG. 1 is an exploded, sectional view of an ear-cleaning implement according to one embodiment thereof; and

FIG. 2 is a perspective view of an assembled ear-cleaning implement.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

In one embodiment, there is disclosed an ear-cleaning implement 10 with an applicator 12 for inserting into an ear canal. Optionally the applicator 12 may be made from material that exhibits shape memory characteristics. The applicator 12 has a distal end region 14, a proximal end region 16 and a compliant middle region 18 therebetween. Preferably the compliant middle region 18 is generally frusto-conical in shape.

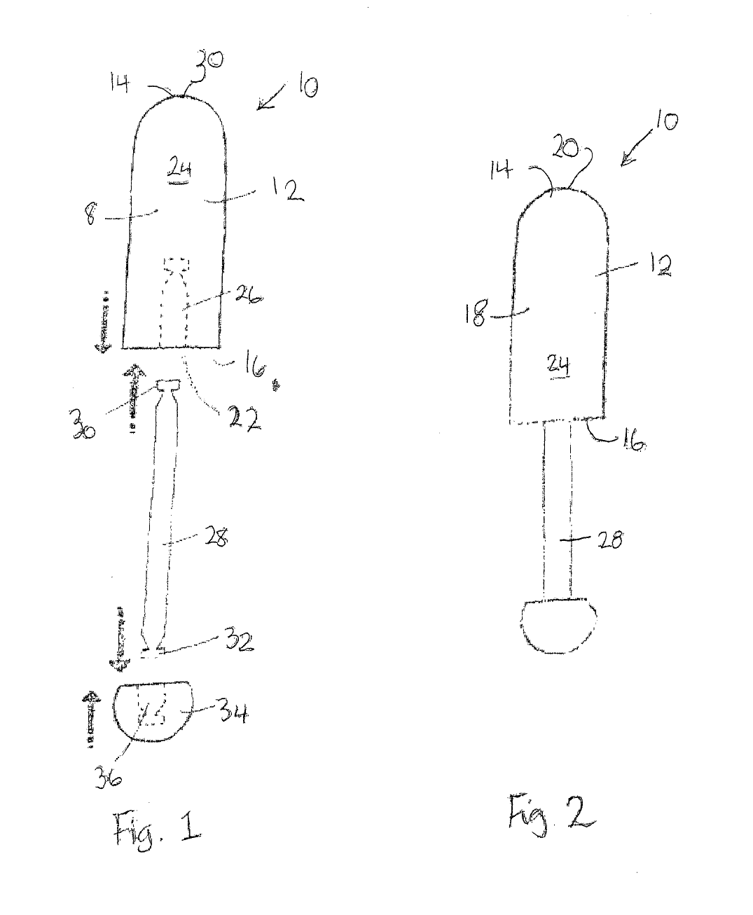

The distal end region 14 is provided with a rounded leading surface 20. At the proximal end region 16 there is an aperture 22 (FIG. 1). If desired the proximal end region 16 may serve as a depth gauge by which the amount of insertion can be visually inspected with the aid of a mirror or by feel.

Between the end regions 14, 16 the compliant middle region 18 has an at rest, un-deflected configuration before insertion into the ear canal. In operation, before the implement 10 is inserted, it is preferably squeezed between the fingers. During passage into the ear canal, after positioning therein, and upon at least partial withdrawal therefrom, the compliant middle region 18 has at least a partially compressed configuration. At least a portion of the compliant middle region 18 conforms with an inner wall of the ear canal and reverts to or towards the un-deflected configuration after compressive forces are removed. Then the applicator 10 is at least partially withdrawn from the ear canal.

Preferably, the applicator 10 has a gentle, non-aggressive adhesive surface 24 that does not impede insertion or cause the implement 10 to bind against the ear wall during insertion. Instead, the adhesive surface 24 guides the implement 10 during passage into the ear canal and withdrawal therefrom. During these positional states, ear wax is gathered on the adhesive surface 24, the wax adhering to at least a portion of the compliant middle region 18 upon withdrawal.

An axial bore 26 is depicted in FIG. 1. It extends from the aperture 22 in the distal end region 16 into the compliant middle region 18. Disposed within the bore 26 is a preferably flexible post 28 that is engaged by the bore 26 so that control can be exercised by a user's fingers in positioning the applicator 10. Another function of the post 28 is to support the compliant middle region 18 and provide some firmness thereto.

In one embodiment, the rounded leading surface 20 of the ear-cleaning implement 10 has a has an absorbent material associated therewith.

In one embodiment, the middle region 18 of the applicator 10 includes a material selected from the group consisting of polyurethane, polyvinyl chloride and mixtures thereof.

In one embodiment, the post 28 has a leading end 30 and a trailing end 32, the leading end 30 optionally including distal means for affixing, such as a barb for engaging the bore 26 so the post 28 and the compliant middle region 18 move in unison. Alternatively, a glue or cement may be used to secure the post 28 within the bore 26.

In one embodiment, the trailing end 32 of the post 28 supports an absorbent compliant rounded surface 34 for cleaning an outer portion of the ear. A proximal means for affixing such as a male-female barb pairing 36 optionally engages the surface 34 and the trailing end 32. The proximal means for affixing the trailing end 32 of the post 28 to the surface 34 may optionally include a glue or cement.

The term “shape memory” applicator as used herein includes shape memory polymers. See, e.g., https://en.wikipedia.org/wiki/Shape-memory_polymer, which is incorporated herein by reference.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An ear-cleaning implement comprising:
an applicator for inserting into an ear canal, the applicator having a distal end region, a proximal end region and a compliant middle region therebetween,
the distal end region having a rounded leading surface;
the proximal end region defining an aperture;
the compliant middle region including a body that has an un-deflected configuration before insertion into the ear canal, a compressed configuration during passage into the ear canal, after positioning therein, and upon at least partial withdrawal therefrom;
a characteristic that at least a portion of the compliant middle region reverts to or towards the un-deflected configuration after compressive forces are removed and the compliant middle region expands into substantial conformity with at least a portion of a wall of the ear canal;
an adhesive surface that gathers ear wax initially located in the ear canal, the wax adhering to at least a portion of the compliant middle region upon withdrawal;
an axial bore extending from the aperture in the distal end region into the compliant middle region; and
a post that lies within and is engaged by the bore so that control can be exercised in positioning the applicator.

2. The ear-cleaning implement of claim 1 wherein the rounded leading surface has an absorbent material associated therewith.

3. The ear-cleaning implement of claim 1 wherein the compliant middle region comprises a shape memory material.

4. The ear-cleaning implement of claim 1 wherein the compliant middle region comprises a material selected from the group consisting of polyurethane, polyvinyl chloride and mixtures thereof.

5. The ear-cleaning implement of claim 1 wherein the post has a leading end and a trailing end, the leading end including distal means for affixing so the post and the compliant middle region move in unison.

6. The ear-cleaning implement of claim 1 wherein the compliant middle region is substantially sphero-cylindrical in shape.

7. The ear-cleaning implement of claim 1 wherein the compliant middle region is substantially frusto-conical in shape.

8. The ear-cleaning implement of claim 5, wherein the trailing end of the post supports an absorbent, compliant rounded surface for cleaning an outer portion of the ear.

9. The ear-cleaning implement of claim 8, wherein the trailing end includes a proximal means for affixing the rounded surface to the trailing end of the post.

10. A method for using an ear-cleaning implement comprising the steps of:
providing an applicator for insertion into an ear canal, the applicator having a distal end region, a proximal end region and a compliant middle region therebetween, the distal end region having a rounded leading surface, the proximal end region defining an aperture;
providing the compliant middle region with a body that has an un-deflected configuration before insertion into the ear canal, a compressed configuration during passage into the ear canal, after positioning therein, and upon at least partial withdrawal therefrom, and a characteristic that at least a portion of the compliant middle region reverts to or towards the un-deflected configuration after compressive forces are removed;
providing an adhesive surface to the applicator that gathers ear wax initially located in the ear canal, the wax adhering to at least a portion of the compliant middle region upon withdrawal;
defining an axial bore extending from the aperture in the distal end region into the compliant middle region; and
securing a post within the bore so that the post is engaged by the bore and so that control can be exercised in positioning the applicator.
